(19) United States
(12) Patent Application Publication
(10) Pub. No.: US 2005/0033302 A1
(43) Pub. Date: Feb. 10, 2005

(54) Implant for the dynamic fixation of a corrective osteotomy

(76) Inventor: Erich Frank, Vienna (AT)

Correspondence Address: JACOBSON HOLMAN PLLC, 400 SEVENTH STREET N.W., SUITE 600, WASHINGTON, DC, 20004, US

(21) Appl. No.: 10/937,476

(22) Filed: Sep. 10, 2004

Related U.S. Application Data
(63) Continuation of application No. PCT/AT03/00067, filed on Mar. 11, 2003.

(30) Foreign Application Priority Data
Mar. 12, 2002 (AT) A 382/2002

(51) Int. Cl. A61B 17/56

(52) U.S. Cl. 606/72

(57) ABSTRACT

In an implant for the dynamic fixation of a corrective osteotomy, it is provided that a fixing section (3) including a hole (9) for the reception of a spongiosa screw is connected via a crank (4) with a particularly laterally offset, elongated anchoring section (1) designed in the longitudinal direction as a rigid anchoring shaft (1), which is preferably configured to include at least three external anchoring edges (12) capable of engaging the inner side of a long bone, thus providing an implant with an improved fixation and a larger corrective range.

The present application is a continuation application of PCT/AT03/00067, filed on Mar. 11, 2003.

### FIELD OF THE INVENTION

The present invention relates to an implant for the dynamic fixation of a corrective osteotomy, wherein a fixing section including a hole for the reception of a spongiosa screw is connected via a crank with a particularly laterally offset, elongated anchoring section designed in the longitudinal direction as a rigid anchoring shaft.

An exemplary, special field of application of such implants comprises corrective osteotomy for the treatment of axial malalignments on metatarsal bones in man, such as, for instance, halux valgus.

### DESCRIPTION OF THE PRIOR ART

From WO 97/35528, an implant designed as a V-shaped brace is known for the treatment of an axial malalignment of a metatarsal bone. That brace does not allow for a completely rotationally stable fixation of the bone fragments to be connected. This involves disadvantages, inter alia, with the foot stressed under load. Furthermore, the insertion of the brace is difficult, because the bone fragment to be displaced will be under chordal tension in most cases. Breakage of the brace may, moreover, occur during explantation.

From U.S. Pat. No. 5,603,715, an implant of the initially defined kind has become known, whereby it is aimed to provide both static and dynamic locking for the treatment of obliquely or transversely extending bone fractures. In addition, an osteosynthesis nail that can be produced from a metallic sheet metal or by casting from a high-strength synthetic material has become known from FR-A 2 572 274.

### SUMMARY OF THE INVENTION

It is the object of the present invention to provide an improved implant for the dynamic fixation of a corrective osteotomy.

To solve this object, an implant for the dynamic fixation of a corrective osteotomy is essentially characterized in that the anchoring shaft is designed to include at least three external anchoring edges capable of engaging the inner side of a long bone.

By this configuration, it is particularly easy to introduce the implant by its shaft into the marrow space of a bone, whereby the anchoring edges will bear against the inner side of the bone and allow for a rotationally fast fixation of the implant. This configuration also enables the bone fragments to be sufficiently displaced and precisely positioned.

When using the implant according to the invention for the dynamic fixation of a corrective osteotomy, the provision of the crank between the fixing section and the anchoring section, in particular, enables a greater dislocation or shift between separated bone parts or fragments such that larger corrections will be feasible by a single cut, for which hitherto several cuts have, for instance, been required. Furthermore, the use of the implant according to the invention, in addition to enabling a greater dislocation as a function of the size of the crank, and hence an improved correction, also directly provides for an accordingly enhanced rotability of the joint surface by applying the cut remotely to the base so as to enable, again by a single cut, both a dislocation by a greater extent than in the prior art and a rotation of the joint surface, to which end two cuts involving accordingly elevated operative expenditures have usually been required according to the prior art known so far.

By using the implant according to the invention, which comprises an anchoring shaft rigid in the longitudinal direction and is preferably provided with anchoring edges for fixation to the inner side of a long bone, even the operative procedure can be shortened and simplified by minimal invasive surgery, since the expansion of the cut will, for instance, be effected by the insertion or introduction of the anchoring shaft between the bone fragments after the application of a shortened cut length in the region of the bone to be separated and after the separation or cutting of the same, whereupon the anchoring shaft is immediately inserted or driven into the long bone after the expansion of the cut and will be immediately firmly anchored within the long bone via the preferably provided anchoring edges such that no additional cumbersome steps will be required for the fixation of the implant, apart from the simple fixation via the spongiosa screw in the region of the fixing section.

To enable particularly simple manufacture while obtaining an accordingly stable implant, it is provided according to a further preferred embodiment that the fixing section, the crank and the anchoring section are designed in one piece.

According to a further preferred characteristic feature of the invention, an anchoring shaft having a T-shaped cross section and a tapered drive-in end may be provided, which anchoring shaft is comprised of a plane, blade-shaped anchoring plate and a web projecting from the same and tapering towards the drive-in end, the side edges of the anchoring plate and the upper edge of the web being designed as anchoring edges.

In accordance with the invention, at least one of the anchoring edges may preferably be sharpened or knife-shaped in the region of the drive-in end.

Furthermore, the shift between the anchoring plate and the fixing section, i.e., the height of the crank, may be selected to be adapted to the desired extent of the lateralization of the bone fragment.

According to a further, preferred characteristic feature of the invention, the hole provided in the fixing section may be designed as a cylindrical bore which, together with the head-near, cylindrical end of the spongiosa screw, forms an angularly stable connection between the spongiosa screw and the implant. Thus, also the angular stability between the bone fragments will be safeguarded.

For a particularly simple fixation of the fixing section, the implant according to the invention is preferably designed in a manner that the hole of the fixing section is designed as a cylindrical bore which, together with the head-near, cylindrical end of the spongiosa screw, forms an angularly stable connection between the spongiosa screw and the implant.

In order to enhance bone formation growth after the insertion of the implant, and hence improve the healing progress, and to enable simplified handling during its introduction into the interior of the long bone, it is proposed according to a further preferred embodiment that the crank is designed to include a diminution or reduction in its lateral regions. By providing such a diminution or reduction, the implant may also be readily removed after a period of, for instance, three to four months, whereby sufficient bone formation to ensure consolidation between the individual bone parts or fragments will be safeguarded particularly in the region of reduction.

For a particularly simple manufacture, it is proposed according to a further preferred embodiment that it is made in one piece of metal. Such metals or metal alloys well tested for use as implants for quite some time are known. Particularly where no removal of the implant is to be effected in order to increase the stability of the connection between the separated bone parts, it is proposed according to a further preferred embodiment of the invention that it is made of resorbable plastics.

### SHORT DESCRIPTION OF THE DRAWINGS

In the following, the invention will be explained in more detail by way of exemplary embodiments illustrated in the drawings. In the drawings,

FIG. 1 shows an osteosynthesis using an implant according to the invention to correct a halux valgus.

FIG. 2 illustrates an implant according to the invention from the side.

FIG. 3 is a top view on the implant of FIG. 2.

FIG. 4 represents a cross section through the implant of FIG. 2 along line IV-IV.

FIG. 5 is a top view similar to FIG. 3 on a modified embodiment of an implant according to the invention.

### DETAILED DESCRIPTION OF PREFERRED EMBODIMENTS

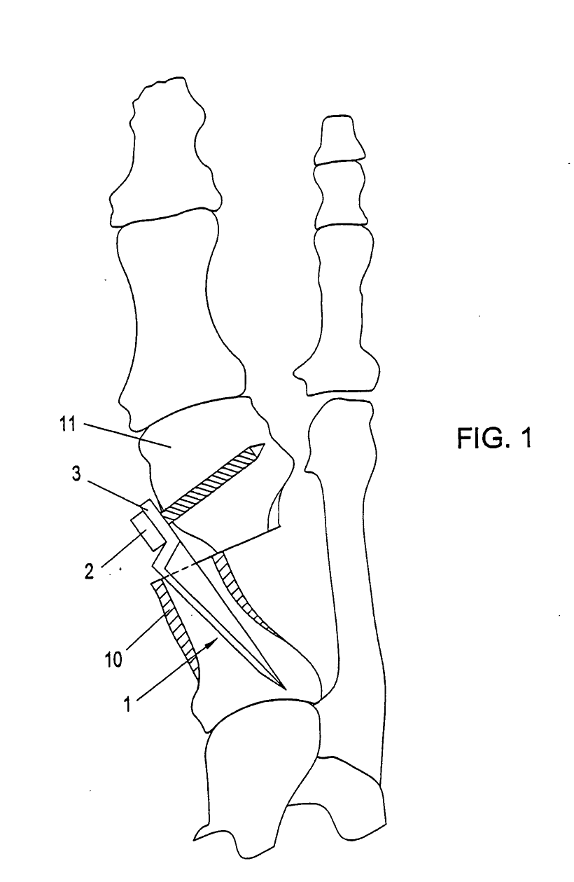

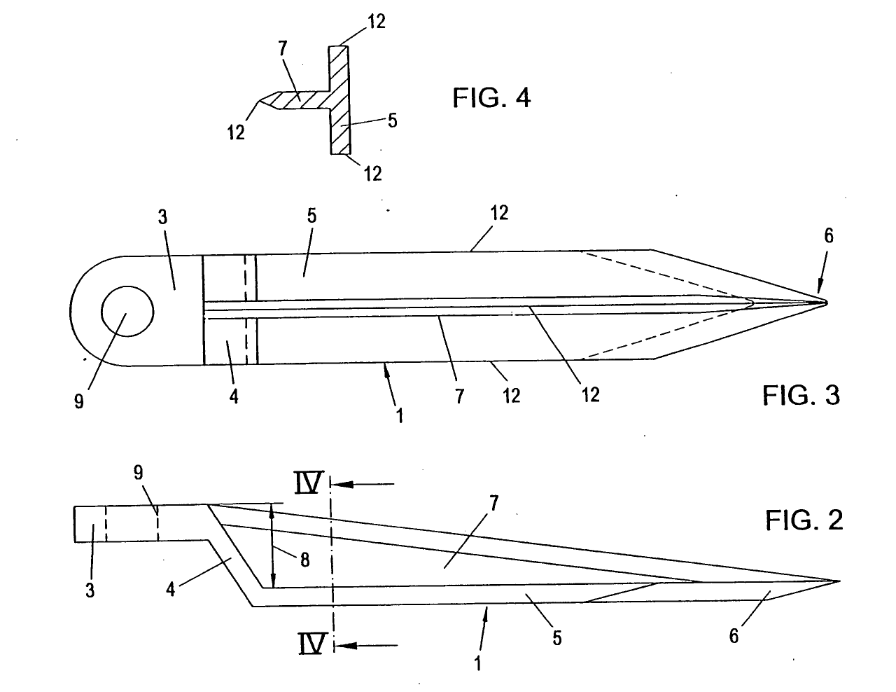

A way of surgically correcting halux valgus consists in separating the malaligned metatarsal I and fixing same in the corrected position. In FIG. 1, such a fixation by the aid of the implant according to FIG. 2 is illustrated. After having separated the bone, the anchoring shaft 1 with the sharpened drive-in end is driven into the marrow space of the bone fragment 10 until the crank comes to lie on the level of the osteotomy. After this, the second bone fragment 11 is fixed to the fixing section 3 by the aid of a spongiosa screw 1. The implant enables a corrective osteotomy that is stable in the frontal and transverse planes as well as in terms of rotation and which allows for the dynamic compression of the bone fragments in the axial direction. The implant stands out for its high static stability. Fracture during explantation need not be feared.

FIG. 2 illustrates an embodiment of the implant from the side. The implant is comprised of an anchoring shaft 1, which is connected with a plate-shaped fixing section 3 via a crank 4. The anchoring shaft 1 is comprised of a plane, edge-shaped anchoring plate 5 and a web 7 upwardly projection from the same and tapering forwards to the drive-in end 6, from which the T-shaped cross section depicted in FIG. 4 results. The lateral edges of the anchoring plate and the upper edge of the web are provided as anchoring edges 12 to be brought into engagement with the inner sides of the long bone. The anchoring plate 5 and the fixing section 3 are located in substantially parallel planes. The height 8 of the crank 4 may be selected to be adapted to the desired displacement of the bone fragment.

The fixing section 3 includes a hole 9 for the reception of a screw 2. This hole may be designed as a cylindrical bore that engages the head-near, cylindrical end of a spongiosa screw, thus enabling the desired angularly stable connection between the screw and the implant.

In a further embodiment of the implant, a fixing section offset further upwards relative to the anchoring plate 5 may be provided on the rear end of the anchoring shaft 1, the crank 4 protruding upwardly beyond the wedge.

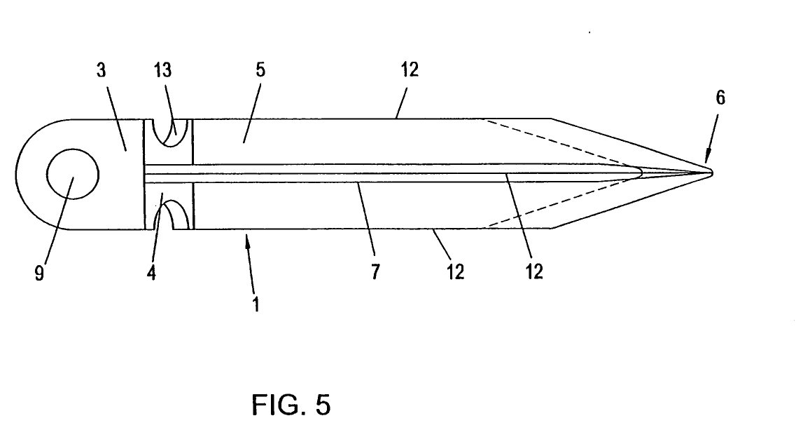

FIG. 5 depicts a modified embodiment, in which the crank 4 is designed to include a reduction or taper or narrowing 13 each in the region of its lateral edges or side edges. In the region of this taper or reduction 13, an improved bone formation, and hence an enhanced stabilization between the fragments 10 and 11, will thus be reached between the mutually offset bone parts or fragments 10 and 11, said reduction 13 also improving or facilitating the handling and subsequent removal of the implant.

The implant can be made of metal or resorbable plastics.

1. An implant for the dynamic fixation of a corrective osteotomy, wherein a fixing section including a hole for the reception of a spongiosa screw is connected via a crank with a particularly laterally offset, elongated anchoring section designed in the longitudinal direction as a rigid anchoring shaft, wherein the anchoring shaft is designed to include at least three external anchoring edges capable of engaging the inner side of a long bone.

2. An implant according to claim 1, wherein the fixing section, the crank and the anchoring section are designed in one piece.

3. An implant according to claim 1, wherein an anchoring shaft having a T-shaped cross section and a tapered drive-in end is provided, which is comprised of a plane, blade-shaped anchoring plate and a web projecting from the same and tapering towards the drive-in end, the side edges of the anchoring plate and the upper edge of the web being designed as anchoring edges.

4. An implant according to claim 1, wherein the hole provided in the fixing section is designed as a cylindrical bore which, together with the head-near, cylindrical end of the spongiosa screw, forms an angularly stable connection between the spongiosa screw and the implant.

5. An implant according to claim 1, wherein at least one of the anchoring edges is knife-shaped, at least in the region of the drive-in end.

6. An implant according to claim 1, wherein the crank is designed to include a diminution or reduction in its lateral regions.

7. An implant according to claim 1, wherein is designed in one piece of metal.

8. An implant according to claim 1, wherein it is made of resorbable plastics.
